(19) United States
(12) Patent Application Publication
(10) Pub. No.: US 2025/0177624 A1
(43) Pub. Date: Jun. 5, 2025

(54) DIVERTER FOR MEMBRANE OXYGENATION DEVICE

(71) Applicant: CHINABRIDGE (SHENZHEN) MEDICAL TECHNOLOGY CO., LTD., Shenzhen (CN)

(72) Inventor: Yijiang Li, Shenzhen (CN)

(21) Appl. No.: 18/968,935

(22) Filed: Dec. 4, 2024

(30) Foreign Application Priority Data
Dec. 1, 2023 (CN) 202311637760.9

(51) Int. Cl. A61M 1/16

(52) U.S. Cl. CPC A61M 1/1698

(57) ABSTRACT

The embodiment of the present application provides a membrane oxygenation device, including a shell, a diverter column is arranged in the shell, a heat exchange part and an oxygenation part are arranged between the diverter column and the shell from the inside to the outside, and a blood inlet and a blood outlet are arranged on the shell; a connector is arranged above the diverter column, and the connector and the diverter column are connected by a plurality of spaced connectors, blood flows into the shell from the blood inlet and is diverted by the diverter column, and then flows into the heat exchange part along the circumferential direction of the shell. The present application can solve the problem of uneven blood flow rate caused by a single outlet in conventional vertical oxygenators.

### CROSS REFERENCE TO RELATED APPLICATION

The present application claims priority to Chinese Patent Application No. 202311637760.9, filed Dec. 1, 2023, the entire disclosure which is hereby incorporated by referenced.

### TECHNICAL FIELD

The embodiments of this application belong to the field of medical devices, and specifically relate to a membrane oxygenation device.

### BACKGROUND

Extracorporeal membrane oxygenation (ECMO) is used to treat patients with cardiopulmonary failure. The core parts of extracorporeal membrane oxygenation (ECMO) are artificial lungs (also called membrane lungs or oxygenators) and artificial hearts (also called blood pumps or power pumps). Cylindrical oxygenation devices have been widely used in the market. The existing cylindrical oxygenators are prone to thrombosis on the opposite side of the blood outlet of the oxygenator due to the structural design of the shunt column.

### SUMMARY

In order to solve or alleviate the problems existing in the prior art, the present application provides a membrane oxygenation device, characterized in that it includes a shell, a diverter column is arranged in the shell, a heat exchange part and an oxygenation part are arranged between the diverter column and the shell from the inside to the outside, and a blood inlet and a blood outlet are arranged on the shell;

A connector is arranged above the diverter column, and the connector and the diverter column are connected by a plurality of spaced connectors. Blood flows into the shell from the blood inlet and is diverted by the diverter column, and then flows into the heat exchange part along the circumferential direction of the shell. The heat exchange part heats the blood, and then the blood enters the oxygenation part for oxygenation, and finally flows out of the shell from the blood outlet;

The upper end surface of the diverter column includes a first upper end surface and a second upper end surface arranged adjacently, the first upper end surface is close to the blood outlet of the oxygenation device, and the second upper end surface is far away from the blood outlet of the oxygenation device, the first upper end surface is a plane, and the second upper end surface is an oblique cut surface.

As an embodiment of the present application, the intersection of the first upper end face and the second upper end face forms an upper tangent of the second upper end face, the upper tangent of the second upper end face is perpendicular to the radial direction of the blood outlet of the oxygenation device, and the bottom of the lower tangent of the second upper end face is set on the side of the diverter column away from the blood outlet.

As an embodiment of the present application, the diameter of the circle where the first upper end face is located is L1, the height of the diverter column is H, the upper tangent is set at a position between 1/2L1 and 3/4L1 away from the blood outlet, and the lower tangent 202b is set at a position between 1/2H and 3/4H away from the blood outlet.

As an embodiment of the present application, the center point of the circle where the first upper end face of the diverter column is located is on the same straight line as the center point of the lower end face of the diverter column, and the difference in diameter between the first upper end face of the diverter column and the lower end face of the diverter column is greater than or equal to 2.5 mm and less than or equal to 5 mm.

As an embodiment of the present application, a plurality of the connectors are spaced and distributed on the edge of the first upper end face and/or the edge of the second upper end face of the diverter column near one end of the diverter column.

As an embodiment of the present application, the connectors located on the edge of the first upper end face of the diverter column are symmetrically arranged on both sides of the first line perpendicular to the upper tangent of the second upper end face and/or arranged on the end point of the first line away from the upper tangent of the second upper end face.

As an embodiment of the present application, the connectors located on the edge of the second upper end face are symmetrically arranged on both sides of the second line perpendicular to the upper tangent of the second upper end face and/or arranged on the end point of the second line away from the upper tangent of the second upper end face.

As an embodiment of the present application, the width of the connector located on the second upper end face of the diverter column is greater than the width of the connector located on the first upper end face of the diverter column.

As an embodiment of the present application, a guide grid is arranged between the heat exchange part and the oxygenation part, and the guide grid is used to guide blood from the heat exchange part to the oxygenation part.

As an embodiment of the present application, the first area, the second area and the third area are separated axially by the guide grid, and the second area has two blocks, and each second area is adjacent to the first area and the second area respectively;

Through holes for blood circulation are arranged in sequence in the circumferential direction and axial direction of the guide grid, and in the same circumferential direction, the through holes are successively smaller in the first area, the second area and the third area; the third area is close to the side of the blood outlet.

Compared with the prior art, the embodiment of the present application provides a membrane oxygenation device with a biased structure through a diverter column, so that blood tends to flow more toward the opposite side of the blood outlet, and enhances the blood flow rate on the opposite side of the blood outlet, solving the problem of low oxygenation efficiency and easy hemolysis in some areas caused by uneven internal blood flow caused by the position of the blood outlet of the existing oxygenation device; and by setting the influence of the flow resistance of the guide grid, the oxygen exchange efficiency in the oxygenation part can be improved.

### BRIEF DESCRIPTION OF THE DRAWINGS

The drawings described here are used to provide a further understanding of the present application and constitute a part of the present application. The schematic embodiments of the present application and their descriptions are used to explain the present application and do not constitute an improper limitation of the present application. The following text will describe some specific embodiments of the present application in detail in an exemplary and non-restrictive manner with reference to the accompanying drawings. The same figure marks in the accompanying drawings indicate the same or similar components or parts. It should be understood by those skilled in the art that these drawings are not necessarily drawn to scale. In the accompanying drawings:

FIG. 1 is a schematic diagram of the three-dimensional cross-sectional structure of the membrane oxygenation device of the embodiment of the present application.

FIG. 2 is a schematic diagram of the cross-sectional structure of the membrane oxygenation device of the embodiment of the present application.

FIG. 3 is a schematic diagram of the three-dimensional structure of the diverter column of the embodiment of the present application.

FIG. 4 is a schematic diagram of the cross-sectional structure of the diverter column of the embodiment of the present application.

FIG. 5 is an enlarged view of the structure at A in FIG. 4 of the embodiment of the present application.

FIG. 6 is a schematic diagram of the three-dimensional structure of the diversion grid of the embodiment of the present application.

FIG. 7 is a schematic diagram of the unfolded planar structure of the diversion grid of the embodiment of the present application.

### DETAILED DESCRIPTION

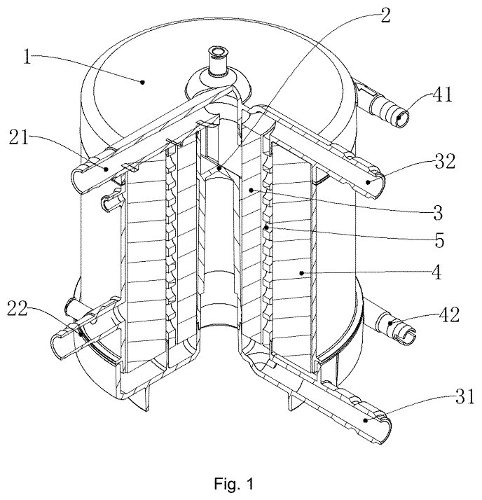

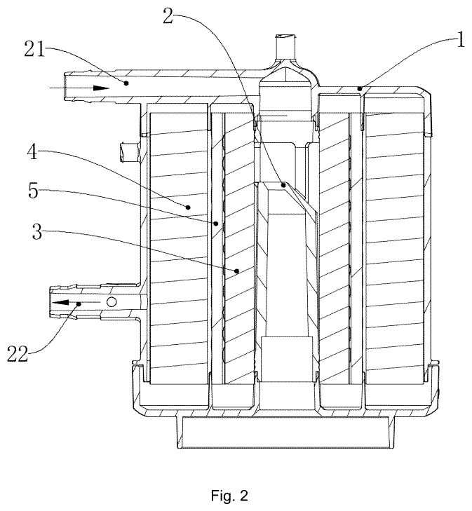

As shown in FIGS. 1-3, the embodiment of the present application provides a membrane oxygenation device, including a shell 1, a diverter column 2 is arranged in the shell 1, a heat exchange part 3 and an oxygenation part 4 are arranged between the diverter column 2 and the shell 1 from the inside to the outside, and a blood inlet 21 and a blood outlet 22 are arranged on the shell 1.

A connector 23 is arranged above the diverter column 2, and the connector 23 and the diverter column 2 are connected by a plurality of spaced connectors 24; blood flows into the shell 1 from the blood inlet 21 and is diverted by the diverter column 2, and then flows into the heat exchange part 3 along the circumferential direction of the shell 1, and the heat exchange part 3 heats the blood, and then the blood enters the oxygenation part 4 for oxygenation, and finally flows out of the shell 1 from the blood outlet 22.

In one embodiment, a hollow heat transfer membrane is arranged inside the heat exchange part 3, and the upper and lower ends of the heat exchange part 3 are respectively connected to the water outlet 32 and the water inlet 31, and the heat transfer medium flows through the heat exchange part to exchange heat with the blood so as to heat the blood, and the heat transfer medium can be water.

In this embodiment, the hollow heat transfer membrane is a material with a special structure, usually composed of a layer of air or other low heat conductivity medium sandwiched between two layers of film, which can provide thermal isolation and energy saving effects during heat transfer. In this application, the blood can be heated so that the temperature of the blood is consistent with the blood temperature inside the human body.

In one embodiment, a hollow oxygenation fiber membrane is installed inside the oxygenation part 4, and the upper and lower ends of the oxygenation part 4 are respectively connected to the air inlet 41 and the air outlet 42, and oxygen flows through the oxygenation part 4 to oxygenate the blood.

In this embodiment, the hollow oxygenation fiber membrane, through its special structure, fully contacts oxygen with red blood cells in the blood, so that gas exchange can be carried out in the oxygenation device. Through the small holes on the oxygenation membrane, oxygen is transferred from the oxygenation device to the blood, and carbon dioxide in the body is discharged at the same time, so that the blood is effectively oxygenated; in order to ensure the safe circulation of blood in the oxygenation device, the hollow oxygenation fiber membrane usually also uses microporous filtration technology and has the function of aseptic isolation, which can prevent microbial contamination and thrombosis and maintain the purity of blood during the circulation process; in addition, the hollow oxygenation fiber membrane has good biocompatibility, can reduce the adverse effects on blood cells and plasma, and reduce the risk of adverse reactions such as coagulation and inflammation.

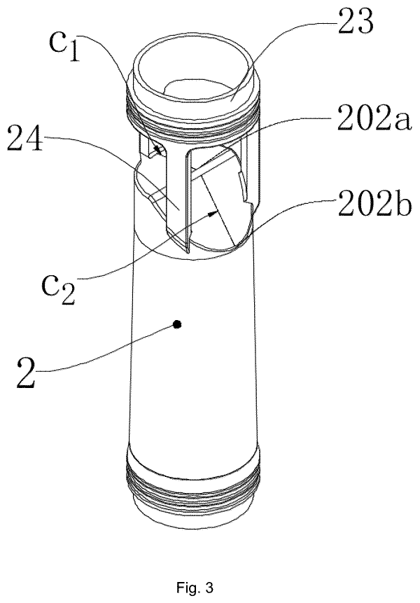

As shown in FIG. 3, the upper end surface includes a first upper end surface 201 and a second upper end surface 202, the first upper end surface 201 is close to the blood outlet 22 of the oxygenation device, and the second upper end surface 202 is far away from the blood outlet 22 of the oxygenation device; the first upper end surface 201 is a plane, and the second upper end surface 202 is an oblique surface.

In certain embodiments, the second upper end surface 202 may also be an arc-shaped slope structure. This embodiment of the present application does not limit this.

In the embodiment of the present application, the first upper end surface 201 and the second upper end surface 202 may have the same area, which is not limited in this embodiment of the present application.

As an embodiment of the present application, the intersection of the first upper end surface 201 and the second upper end surface 202 forms an upper tangent 202a of the second upper end surface 202, the upper tangent 202a is perpendicular to the radial direction of the blood outlet 22 of the oxygenation device, and the bottom of the lower tangent 202b is arranged on the side of the diverter column 2 away from the blood outlet 22.

In this embodiment, the structure of the traditional shunt column 2 is improved. Specifically, the present application sets a second upper end face 202 on the shunt column 2, and the second upper end face 202 is used to bias the blood to the opposite side of the blood outlet 22 of the oxygenation device. The blood forms a negative pressure at the second upper end face 202, so that the blood can be more inclined to flow into the opposite side of the blood outlet, and the blood flow rate on the opposite side of the blood outlet 22 is enhanced, which solves the technical problem that thrombus is easy to appear on the opposite side of the blood outlet 22 of the existing oxygenation device.

As shown in FIG. 3, in one embodiment, a plurality of connectors 24 are spaced and distributed on the edge of the first upper end face 201 and/or the edge of the second upper end face 202 of the shunt column 2 near one end of the shunt column 2.

By setting the connector 24 between the shunt column 2 and the connector 23, on the one hand, the connector 24 can make the connector 23 stably installed on the shunt column 2, and on the other hand, the setting of the connector 24 can also achieve the function of diversion, and can divert the blood to the side of the shunt column 2 away from the blood inlet 21, further increasing the diversion effect of the shunt column 2.

In one embodiment, the connector 24 located on the edge of the first upper end surface 201 of the diverter column 2 is symmetrically arranged on both sides of the first line C1 perpendicular to the upper tangent line 202a and/or is arranged at the end point of the first line C1 away from the upper tangent line 202a.

In one embodiment, the connector 24 located on the edge of the second upper end surface 202 is symmetrically arranged on both sides of the second line C2 perpendicular to the upper tangent line 202a and/or the connector 24 is arranged away from the upper tangent line. Set at the end point of the second line C2 away from the upper tangent line 202a.

In this embodiment, the connector 24 is symmetrically arranged on the second upper end surface 202, so that the connector 24 can cooperate with the second upper end surface 202 to achieve a better drainage effect and more effectively prevent the formation of thrombus.

In one embodiment, the width of the connector 24 located on the edge of the diverter column 2 is greater than the width of the connector 24 located on the upper end surface 201 of the diverter column 2.

In this embodiment, by setting the width of the connector 24 on the bevel edge of the diverter column 2 greater than the width of the connector 24 on the upper end surface 201, the drainage effect of the connector 24 on the diverter column 2 is better, and the embodiment of the present application can also make the diverter column 2 more stable.

In one embodiment, there are at least two connectors 24, and several connectors 24 are evenly distributed on the edge of the first upper end surface 201 and/or the edge of the second upper end surface 202. When there are two connectors 24, two connectors 24 are symmetrically arranged on the second upper end surface 202. When there are four connectors 24, four connectors 24 are arranged in groups of two, and are symmetrically arranged on the first upper end surface 201 and the second upper end surface 202 respectively;

In certain embodiments, three connectors 24 are arranged, and the three connectors 24 can form a triangular distribution, which can improve stability while not affecting blood flow.

In one embodiment, one connector 24 is arranged at the end point of the first line C1 away from the upper tangent 202a, and the other two connectors 24 are arranged on both sides of the second line C2 perpendicular to the upper tangent 202a of the diversion column 2; or,

Two connectors 24 are arranged at the end point of the first line C1 away from the upper tangent 202a, and the other connector 24 is arranged on both sides of the second line C2 perpendicular to the upper tangent 202a.

In one embodiment, a smooth portion 203 is provided at the connection between the first upper end surface 201 and the second upper end surface 202.

In this embodiment, the design of the smooth portion 203 can better guide blood, reduce resistance and improve the flow performance of blood compared to acute angle or right angle connection, and can better guide blood so that blood flows more smoothly in the connection area.

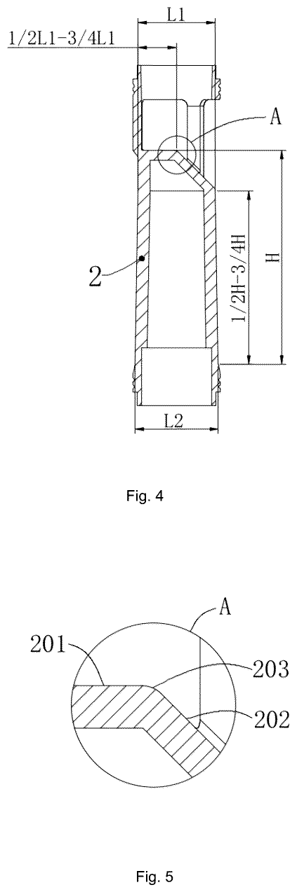

As shown in FIG. 4, in one embodiment, the diameter length of the circle where the first upper end surface 201 of the diverter column 2 is located is L1, the height of the diverter column 2 is H, the upper tangent 202a is set at a position between 1/2L1 and 3/4L1 away from the radial direction of the blood outlet 22, and the lower tangent 202b is set at a position between 1/2H and 3/4H away from the blood outlet 22.

In certain embodiments, the length of the upper tangent 202a does not exceed the diameter length of the first upper end surface 201, and the lowest point of the lower tangent 202b is not lower than the position where half of the height of the diverter column 2 is located.

In this embodiment, the length of the upper tangent line 202a does not exceed the diameter length of the first upper end surface 201, and the lowest point of the lower tangent line 202b is not lower than the position where half of the height of the shunt column 2 is located, so that the size of the formed slope can be ensured to be moderate, so that the blood on the opposite side of the blood outlet 22 of the oxygenation device will not cause thrombosis.

In one embodiment, H=4 L1.

In one embodiment, the length of the upper tangent line 202a is between 25 and 30 mm.

In this embodiment, it can be ensured that the size of the formed slope is moderate, so that the blood on the opposite side of the blood outlet 22 of the membrane oxygenation device will not cause thrombosis.

In one embodiment, the shunt column 2 is a conical structure or a cylindrical structure.

In a specific embodiment, the cross-section of the shunt column 2 gradually increases from top to bottom, because more blood begins to flow into the upper end of the shunt column 2, so as to avoid the accumulation of blood on the upper end of the shunt column 2. The shunt column 2 can be a conical structure or a cylindrical structure, and the embodiment of the present application does not limit this.

In one embodiment, the center point of the circle where the first upper end surface 201 of the diverter column 2 is located is on the same straight line as the center point of the circle where the lower end surface of the diverter column 2 is located. The length of the straight line passing through the center point of the lower end surface and parallel to the upper tangent 202a is L2, and the difference between L2/2 and L1/2 is greater than or equal to 2.5 mm and less than or equal to 5 mm. In certain embodiments, the difference between L2/2 and L1/2 is 3 mm.

As shown in FIG. 5, in one embodiment, the smooth portion 203 is a smooth arc surface.

In this embodiment, the design of setting the smooth portion 203 as a smooth arc surface can reduce the turbulence and resistance generated by blood in the connection area. Compared with acute angle or right angle connection, the smooth arc surface can better guide blood, reduce resistance and improve the flow performance of blood. The curvature of the arc surface can better guide blood, so that blood flows more smoothly in the connection area. It helps to reduce blood separation and pressure loss, thereby improving the diversion effect.

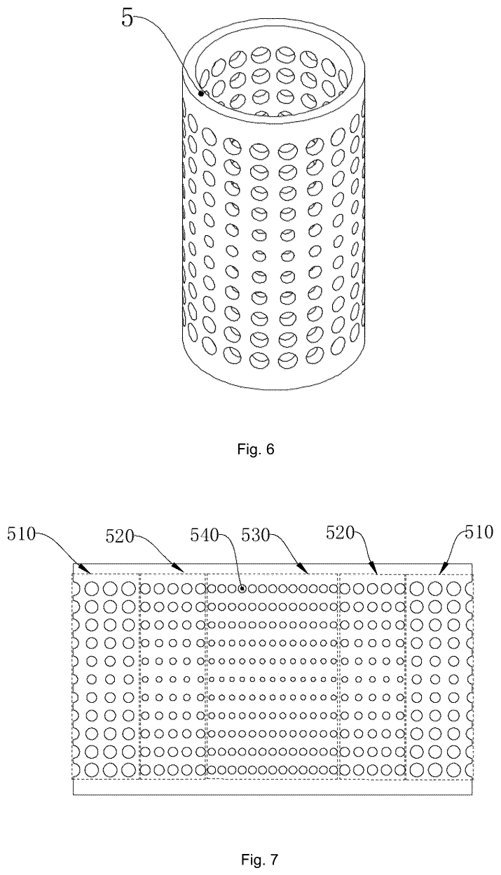

As shown in FIG. 6, in one embodiment, a flow guide grid 5 is provided between the heat exchange part 3 and the oxygenation part 4, and the flow guide grid 5 is used to guide blood from the heat exchange part 3 to the oxygenation part 4.

In this embodiment, the position of the flow guide grid 5 is optimal. By comparison, the flow guide grid 5 provided between the heat exchange part 3 and the oxygenation part 4 can achieve the best oxygenation effect.

As shown in FIG. 7, in one embodiment, the flow guide grid 5 is axially separated into a first area 510, a second area 520 and a third area 530, and the second area 520 has two blocks, and each second area 520 is adjacent to the first area 510 and the third area 530 respectively;

Through holes 540 for blood circulation are arranged in sequence in the circumferential direction and axial direction of the flow guide grid 5, and in the same circumferential direction, the through holes 540 are successively smaller in the first area 510, the second area 520 and the third area 530; the third area 530 is close to the side of the blood outlet 22.

In this embodiment, the through holes 540 on the same axial direction of the guide grid 5 decrease from top to bottom and then increase in sequence, and the through holes 540 at both ends of the same axial direction are of the same size.

In order to prevent the blood from being retained for too long through the through holes 540 on the guide grid 5, the through holes 540 at both ends of the same axial direction are of the same size in any of the first area 510, the second area 520 and the third area 530, and the through holes 540 decrease and then increase in sequence from top to bottom. The purpose of doing so is to take into account that the blood pressure increases with the depth of the blood in the through holes 540 on the same axial direction. In other words, the through holes 540 at the upper end of the guide grid 5 have a small flow rate and are prone to thrombosis.

However, if the size of the through holes 540 at the lower end of the guide grid 5 is set to the minimum, it is also easy to form a slow flow zone at the bottom of the shell, leading to the formation of thrombus. By setting the middle through hole 540 on the same axial direction to the minimum, the formation of thrombus can be greatly reduced. Because the blood flowing out of the through holes 540 above and below it will drive the blood flowing out of the smallest through hole 540 in the middle to flow together.

Generally speaking, there are through holes 540 in the same axial direction, and the smallest through hole 540 is located in the middle of this row of through holes 540.

In an embodiment, the first area 510, the second area 520 and the third area 530 are of the same size. In this embodiment, the first area 510, the second area 520 and the third area 530 are of the same size, which can be easily processed to ensure that the specifications of each guide grid 5 are consistent.

In an embodiment, the blood outlet 22 is close to the central axis of the third area 530 in the axial direction of the guide grid 5 and the central axis of the blood outlet 22. In this embodiment, after the guide grid 5 is installed, the central axis of the third area 530 in the longitudinal direction is the central axis of the blood outlet 22. That is to say, the blood outlet 22 is directly opposite to the central axis of the third area 530. This ensures that the blood flow state at both ends with the central axis of the third area 530 as the dividing interface remains basically symmetrical. In this way, it can be ensured that the blood at any point will not be retained for too long to form a thrombus due to the difference in flow rate.

In an embodiment, the through hole 540 is circular. It should be noted that the shape of the through hole 540 can be a rhombus, a square, an ellipse or other shapes, for example a circle. In certain embodiments, the through hole 540 is a circular hole with a rounded arc.

In an embodiment, the center distance between two adjacent through holes 540 is 4-9 mm. In this embodiment, the two adjacent through holes 540 include the positional relationship of up, down, left and right. The center distance between two adjacent through holes 540 is the same, and the value range is 4-9 mm. This can ensure that the blood is better guided after passing through the through hole 540 to avoid thrombus.

In an embodiment, the maximum diameter of the through hole 540 in the first area 510 is 5.5-6.5 mm, and the minimum diameter is 3.5-4.5 mm; the maximum diameter of the through hole 540 in the second area 520 is 4-5 mm, and the minimum diameter is 2-3 mm; the maximum diameter of the through hole 540 in the third area 530 is 3-4 mm, and the minimum diameter is 1.5-2 mm.

In one embodiment, the wall thickness of the guide grid 5 is 6-10 mm. In this embodiment, in order to make the guide grid 5 have a better guide effect, the guide grid 5 needs to have a certain thickness, so that the through holes on the guide grid 5 form a channel, which can achieve a better directional guide effect. In certain embodiments, the wall thickness of the guide grid 5 is 6-10 mm.

In one embodiment, after entering the oxygenator, the blood first passes through the heat exchange part 3, and then passes through the oxygenation part 4 to flow to the blood outlet. In the heat exchange part 3, due to the obstruction of the guide grid 5 and the variable temperature membrane, especially the guide grid 5 near the blood outlet has a smaller aperture and a larger resistance, and the guide grid 5 far away from the blood outlet has a larger aperture and a smaller resistance. On the one hand, the flow blocking effect of the guide grid 5 makes the blood flow through the oxygenation part 4 longer than the heat exchange part 3, which increases the oxygenation time of the blood in the oxygenation part 4 and also improves the oxygenation efficiency. On the other hand, according to the design of the holes on the guide grid 5, the blood flow direction can be adjusted to increase part of the resistance in the adaptive direction, so that the blood flow direction of the oxygenation part 4 is more uniform to avoid the situation of blood flow aggregation in some areas, thereby improving the efficiency of blood oxygenation.

In one embodiment, the diverter column 2 is provided with a hub structure connected to the upper cover and lower cover of the oxygenation device on the upper and lower covers, and the hub structure is used to combine with the upper and lower covers of the oxygenator. The upper hub and the head formed by the shunt column 2 are connected by a connector to form a whole, which facilitates the installation and stability of the entire shunt column 2 in the oxygenator housing.

Compared with the prior art, the embodiment of the present application provides a membrane oxygenation device, which can make blood tend to the opposite side of the blood outlet through the radial shunt column, enhance the blood flow rate on the opposite side of the blood outlet, and solve the technical problem that thrombus is easy to appear on the opposite side of the blood outlet of the existing oxygenation device.

Finally, it should be noted that the above embodiments are only used to illustrate the technical solution of the present application, rather than to limit it; although the present application is described in detail with reference to the above embodiments, ordinary technicians in this field should understand that they can still modify the technical solutions recorded in the above embodiments, or replace some or all of the technical features therein by equivalent; and these modifications or replacements do not make the essence of the corresponding technical solution deviate from the scope of the technical solution of the embodiments of the present application.

I claim:

1. A membrane oxygenation device comprising:
a shell, a diverter column is arranged in the shell, a heat exchange part and an oxygenation part are arranged between the diverter column and the shell from the inside to the outside, and a blood inlet and a blood outlet are arranged on the shell;
a connector is arranged above the diverter column, and the connector and the diverter column are connected by a plurality of spaced connectors, blood flows into the shell from the blood inlet and is diverted by the diverter column, and then flows into the heat exchange part along the circumferential direction of the shell, the heat exchange part heats the blood, and then the blood enters the oxygenation part for oxygenation, and finally flows out of the shell from the blood outlet;
wherein the upper end surface of the diverter column includes a first upper end surface and a second upper end surface arranged adjacently, the first upper end surface is close to the blood outlet of the oxygenation device, and the second upper end surface is far away from the blood outlet of the oxygenation device, the first upper end surface is a plane, and the second upper end surface is an oblique cut surface.

2. The membrane oxygenation device of claim 1, wherein the intersection of the first upper end face and the second upper end face forms an upper tangent of the second upper end face, the upper tangent of the second upper end face is perpendicular to the radial direction of the blood outlet of the oxygenation device, and the bottom of the lower tangent of the second upper end face is set on the side of the diverter column away from the blood outlet.

3. The membrane oxygenation device of claim 2, wherein the diameter of the circle where the first upper end face is located is L1, the height of the diverter column is H, the upper tangent is set at a position between 1/2L1 and 3/4L1 away from the radial direction of the blood outlet, and the lower tangent is set at a position between 1/2H and 3/4H away from the blood outlet.

4. The membrane oxygenation device of claim 1, characterized in that the center point of the circle where the first upper end face of the diverter column is located is on the same straight line as the center point of the lower end face of the diverter column, and the difference in diameter between the first upper end face of the diverter column and the lower end face of the diverter column is greater than or equal to 2.5 mm and less than or equal to 5 mm.

5. The membrane oxygenation device of claim 2, wherein a plurality of the connectors are spaced and distributed on the edge of the first upper end face and/or the edge of the second upper end face of the diverter column near one end of the diverter column.

6. The membrane oxygenation device of claim 5 wherein the connectors located on the edge of the first upper end face of the diverter column are symmetrically arranged on both sides of the first line perpendicular to the upper tangent of the second upper end face and/or are arranged on the end point of the first line away from the upper tangent of the second upper end face.

7. The membrane oxygenation device of claim 5 wherein the connectors located on the edge of the second upper end face are symmetrically arranged on both sides of the second line perpendicular to the upper tangent of the second upper end face and/or are arranged on the end point of the second line away from the upper tangent of the second upper end face.

8. The membrane oxygenation device of claim 5 wherein the width of the connector located on the second upper end face of the diverter column is greater than the width of the connector located on the first upper end face of the diverter column.

9. The membrane oxygenation device of claim 1 wherein a guide grid is provided between the heat exchange part and the oxygenation part, and the guide grid is used to guide blood from the heat exchange part to the oxygenation part.

10. The membrane oxygenation device of claim 9 wherein the guide grid is axially separated into a first area, a second area and a third area, and the second area has two blocks, and each second area is adjacent to the first area and the second area respectively; and further wherein through holes for blood circulation are arranged in sequence in the circumferential direction and axial direction of the guide grid, and in the same circumferential direction, the through holes are successively smaller in the first area, the second area and the third area; the third area is close to the side of the blood outlet.
